(19) United States
(12) Patent Application Publication
(10) Pub. No.: US 2020/0040323 A1
(43) Pub. Date: Feb. 6, 2020

(54) Urease Purification And Purified Urease Products Thereof And Sorbent Cartridges, Systems And Methods Using The Same

(71) Applicant: Fresenius Medical Care Holdings, Inc., Waltham, MA (US)

(72) Inventors: Jun Yi, Norman, OK (US); Harshavardhana Reddy Mallipalli, Edmond, OK (US)

(21) Appl. No.: 16/049,954

(22) Filed: Jul. 31, 2018

(51) Int. Cl. C12N 9/80; C12N 11/14; B01J 20/24; B01J 20/32; A61M 1/16; B01D 15/36

(52) U.S. Cl. CPC C12N 9/80; C12Y 305/01005; C12N 11/14; B01J 20/24; B01D 15/363; A61M 1/1696; A61M 1/1656; B01D 15/362; B01J 20/3231

(57) ABSTRACT

Methods for purifying urease are described that make use of a precipitating agent such as ammonium sulfate to obtain urease as a precipitate. The method can involve use of a solubilizing agent, such as a citrate-containing solution, to dissolve the precipitate. The method can involve the use of a sugar to provide protected urease in a sugar-urease solution and immobilization of the urease. The method can involve freeze drying of the immobilized urease. A sugar-urease preparation and an immobilized urease, and a sorbent cartridge containing the urease are described as well as methods to conduct dialysis.

### BACKGROUND OF THE INVENTION

The present invention relates to purifying urease and its use in the manufacture of articles made therewith and use of the articles in purification systems and methods.

Sorbent-based purification is under investigation in diverse areas such as hemodialysis fluid regeneration systems and wastewater treatment. Spent or used dialysate can be regenerated in a sorbent cartridge for reuse in the dialyzer. An important component of sorbent cartridges used in hemodialysis, for example, is the enzyme urease. Urease can be included in a packed bed of sorbent in the sorbent cartridge. Urease hydrolyzes urea waste in spent dialysate to form ammonium and bicarbonate ions, which can be removed by other sorbent materials included in the sorbent bed.

Urease can be commercially obtained as an isolated and purified product derived from natural or synthetic sources. Canavalia ensiformis (“Jack beans”) is a common source of highly active urease. Urease is only a minor component of Jack beans, as most of the bean content is made up of non-urease materials, such as other proteins, lipids, and fats. Known methods of isolating and purifying urease from Jack beans are difficult to scale up, inefficient, or costly and time consuming.

One such method of isolating urease from Jack beans involves extracting protein from milled Jack beans (Jack bean meal), then solid-liquid separating the extract mixture using a filter-press to remove undissolved particles, followed by ultrafiltration (UF), diafiltration (DF) cycles of the supernatant solution using a porous membrane to selectively remove small biomolecules, and then adsorbing urease on a support material and drying the resulting enzyme-containing cake before packing into sorbent cartridges. Scale-up of such a separation method is difficult because of particle size requirements in the industry on milled Jack beans (e.g., fine particles with high enzyme activity cannot be used), high cost of the scale up operations, membrane fouling, unwanted protein aggregation during the UF/DF process which leads to inefficient separation of urease from contaminant proteins (e.g., Concanavalin A) and other undesired biomolecules, and porous membrane fouling problems.

As another method, U.S. Patent Application Publication No. 2016/0032268 describes a method for isolating and purifying urease from natural sources of urease, such as ground Jack bean meal, which comprises defatting the natural sources of urease by mixing them with cold solvent to create a mixture thereof and mechanically separating the mixture, and extracting urease from the defatted natural sources of urease by adding a buffer solution and mechanically separating the resulting mixture, and purifying the resulting urease solution.

In another method, U.S. Pat. No. 3,249,513 describes a process for separating urease from leguminous material which comprises comminuting leguminous material to form a meal, slurrying the meal in water at a pH maintained at about 7.0, separating the solid phase from the aqueous phase and dialyzing the aqueous phase through a cellulosic membrane against a dilute aqueous buffer solution maintained at a pH of about 7.5. The purified urease solution may thereafter be lyophilized or the water present therein may be separated by spray-drying to form a powder.

As another consideration, for use in sorbent cartridges, urease is commonly immobilized so that it can be retained at a fixed location within the cartridge housing as fluid undergoing treatment flows through the cartridge. Jack Bean meal has been immobilized by being blended with filler such as alumina, with the resulting paste formed into a layer included in a sorbent bed housed in a cartridge. See, e.g., U.S. Patent Application No. 2017/0189598 A1, which is incorporated in its entirety by reference herein. In another method, U.S. Pat. No. 4,721,652 describes a mixed type urease particle that can be used in sorbent dialysis, which comprises urease mixed with powders of silica gel, alumina, inorganic porous sintered bodies, or diatomaceous earth and the like, and a cellulose system adhesive added to this mixture, and after mixing is thereafter dried and milled to prepare a preblend urease powder, which can be formed into pellets.

It is desirable to find new methods of urease isolation and/or purification from Jack beans and other urease sources, which are scalable to efficiently produce high purity urease in increased amounts and improved arrangements for immobilizing the high purity urease for use in sorbent cartridges or other purification devices used in the regeneration of used dialysate solutions, wastewater treatment, or other uses.

### SUMMARY OF THE PRESENT INVENTION

A feature of the present invention is to provide a method for obtaining high purity urease which meets the above and/or other needs.

An additional feature of the present invention is to provide highly purified urease products, such as stable urease liquid preparations and solid immobilized urease products containing high purity urease, which can be used in the manufacture of sorbent beds and sorbent cartridges, which are useful for dialysis systems and treatments, wastewater systems and treatments, or other fluid regeneration or purification systems and treatments.

Additional features and advantages of the present invention will be set forth in part in the description that follows, and in part will be apparent from the description, or may be learned by practice of the present invention. The objectives and other advantages of the present invention will be realized and attained by means of the elements and combinations particularly pointed out in the description and appended claims.

To achieve these and other advantages, and in accordance with the purposes of the present invention, as embodied and broadly described herein, the present invention relates, in part, to a method of purifying urease. The method includes a) mechanically separating an extract mixture to provide a separated solution, wherein the extract mixture comprises at least one extracting agent in solution and a comminuted source of urease; b) combining ammonium sulfate with the separated solution to precipitate urease in the separated solution to provide a precipitate-containing mixture; c) mechanically separating the precipitate-containing mixture to collect the precipitate; d) dissolving the collected precipitate to provide a urease-containing solution; e) combining the urease-containing solution with sugar to provide a sugar-urease solution; f) combining the sugar-urease solution and sorbent material to immobilize urease on the sorbent material to provide an immobilized urease preparation; and g) optionally freeze drying the immobilized urease preparation to provide an immobilized urease product.

The present invention further relates to a method of purifying urease, comprising a) combining ammonium sulfate with an urease-solute containing solution to precipitate urease to provide a precipitate-containing mixture; b) mechanically separating the precipitate-containing mixture to collect the precipitate; c) combining the precipitate, a sugar, and a solubilizing agent in solution, in any order, to provide a sugar-urease solution; and d) combining the sugar-urease solution and sorbent material to immobilize urease on the sorbent material to provide an immobilized urease preparation.

The present invention further relates to a sugar-urease liquid preparation, such as prepared by one of the methods described herein.

The present invention further relates to an immobilized urease product comprising sorbent material, urease immobilized on the sorbent material, and sugar, such as the immobilized urease product obtained from one of the methods described herein.

The present invention further relates to a sorbent cartridge comprising a urease-containing layer comprising the immobilized urease product.

The present invention further relates to a method to regenerate or purify dialysis fluid comprising passing dialysis fluid through the sorbent cartridge.

The present invention further relates to a dialysis system to regenerate or purify spent dialysis fluid comprising the sorbent cartridge.

It is to be understood that both the foregoing general description and the following detailed description are exemplary and explanatory only and intended to provide a further explanation of the present invention, as claimed.

### BRIEF DESCRIPTION OF DRAWINGS

FIG. 1 is a process flow diagram of a method of production of purified urease products according to an example of the present application.

FIG. 2 is an exploded view of materials in a sorbent cartridge according to an example of the present application.

FIG. 3 is a schematic diagram showing a sorbent dialysis system which includes a sorbent cartridge according to an example of the present application.

### DETAILED DESCRIPTION OF THE PRESENT INVENTION

The present invention relates to obtaining high purity urease from Jack beans and/or other sources of urease. In a method thereof, mixtures of urease source material(s) and extraction buffer(s) are separated using non-membrane based techniques, and the separated solution is salted out with a precipitating agent. This process can minimize the precipitation of proteins other than urease. After collecting and dissolving the precipitate, the resulting urease-containing solution can be treated with or protected, for instance, by a sugar addition. The sugar can reduce protein-protein interactions and/or protect the native urease during any downstream freeze drying. A sugar-urease solution/preparation can thus be provided which contains protected urease. As used herein, “protecting” urease refers to preserving, stabilizing, and/or ensuring that at least a portion of the enzyme (e.g., at least 50%, at least 75%, or at least 90% by weight) is not denatured or destroyed during lyophilization and/or other process operations applied to a urease-containing material such as those described herein. To produce an immobilized form of the urease, the resulting sugar and urease-containing solution can be mixed with sorbent material to adsorb or otherwise bind or attach the urease enzyme from the solution, and the combined sorbent-urease can be lyophilized or otherwise dried to produce an immobilized urease product, which contains high purity and activity urease. The sugar can preserve activity of urease in lyophilization.

Introduction of sugar into the urease-containing solution upstream of lyophilization in the indicated process can unexpectedly increase efficiency of urease recovery during the freeze drying step, such as by 10% or more or about 25% or more, or about 30% or more, or about 40% or more, or about 50% or more, or about 60% or more, or from about 10% to 75%, or from about 25% to about 75%, or from about 35% to about 65%, or from about 40% to about 60%, or other increases, compared to the same process performed without including sugar addition to treat the urease-containing solution.

Though not desiring to be bound to a particular theory, the sugar protection of the urease in the sugar-urease solution, either itself or in combination with lyophilization, can increase recovery of urease in its native quaternary state, thereby improving the enzymatic activity of the resulting purified urease product. The method can improve activity in the resulting urease, making it possible to reduce the amount of urease used in a cartridge (and/or reducing the size of the cartridge). Smaller sorbent cartridges can be used with use of high purity and activity urease which can be obtained by the present invention. For instance, with the present invention, the amount of urease and/or size of the cartridge can be reduced by 5% or more, such as 10% or more, 15% or more, or even 20% or more. In addition, the method can reduce the amount of sorbent material required to retain the urease on the cartridge. Further, the disclosed methods can readily be scaled up for commercial production, by for example allowing a more continuous purification strategy which is less reliant on batch operations, as detailed further herein.

As a further option for the method, an extractant of milled Jack beans or other urease source in a buffer solution can be centrifuged, and ammonium sulfate can be added to the supernatant of the extract mixture to optimally precipitate urease while minimizing the precipitation of other proteins. The resulting precipitate and supernatant can be centrifuged, and the resulting pellet can be redissolved in buffer solution. Sugar can then be added to the solution to provide a sugar-urease solution (intermediate) or urease preparation product. The resulting sugar-urease solution can then be mixed with a sorbent material to adsorb and immobilize the urease enzyme. The slurry can be filtered and the sorbent-urease cake can be lyophilized in a freeze dryer. The resulting immobilized urease product can be used in sorbent cartridges or other purification devices used for urea removal from aqueous fluids.

The use of centrifugation for non-membrane mechanical solid-liquid separations in the method, in combination with ammonium sulfate precipitation, can be cleaner and more economical than a filter press system combined with UF/DF purification, such as by eliminating the costs for membrane replacement during UF/DF. This option can provide these improvements in a method together with or independent from a downstream step of sugar addition. The combination of the centrifugation, ammonium sulfate precipitation, and the sugar addition steps in the same method can synergize the overall improvements achieved in urease purity and/or yield. Accordingly, the present invention can provide an alternative method of urease purification from Jack beans and other urease sources that can produce high quality urease in good yields as compared to urease isolation and purification strategies using filter presses and membrane filtrations. The methods of the present invention also provide improved methods for binding high purity urease in highly intact form to sorbent material using the sugar-urease preparations to provide improved immobilized urease products for sorbent cartridges. Sorbent cartridges can be provided by the present invention which are cleaner (with regard to urease purity), lighter in weight and/or smaller in dimensions (e.g., less urease and/or other sorbent material(s) is/are needed). These sorbent cartridges can be used in the regeneration of used dialysate solutions, industrial or municipal waste waters, or other aqueous fluids contaminated with urea.

The present invention thus can provide improved urease purification and resulting highly purified urease-containing products. As used herein, “purifying”, “purification”, or “purified” can refer to processes including one or more steps intended to separate urease from non-urease material. These processes can provide urease-enriched materials, preparations, or products, which contain urease at higher concentrations as compared to urease-containing material at an earlier stage of the processing As used herein, “isolating” can refer to initial urease recovery processes, such as extracting, used to separate urease from its natural or synthetic urease source as raw material and/or after the indicated preprocessing (e.g., dehulling, comminuting, defatting, etc.), and yielding an intermediate urease-containing material which can be further purified.

As used herein, “urease” (e.g., urea amidohydrolase, EC 3.5.1.5) catalyzes the hydrolysis of urea, such as to yield ammonia ions and bicarbonate ions. Urease is a multimer protein composed of several 90 kDa monomers. For the present invention, the urease source can be a natural or synthetic source of urease, or both. “Natural sources of urease,” as used herein, refers to any portion of a living or dead organism, including plants, bacteria, animal, algae, or fungi from which urease may be extracted or otherwise separated. Canavalia ensiformis (“Jack beans”) is a natural source of urease. Other natural sources of urease include, but are not limited to, soy beans, sword beans (Canavalia gladiate), bacteria (e.g., H. pylori bacteria, Bacillus pasteurii bacterial), or other plants, algae, bacteria or fungi, and/or invertebrate sources of urease (e.g., ruminal urease). Synthetic urease can be recombinant urease, such as Helicobacter pylori recombinant urease B, recombinant Bacillus subtilis urease, recombinant Bacillus pasteurii urease, or other recombinant ureases. Any source of urease or combination of sources of ureases can be used.

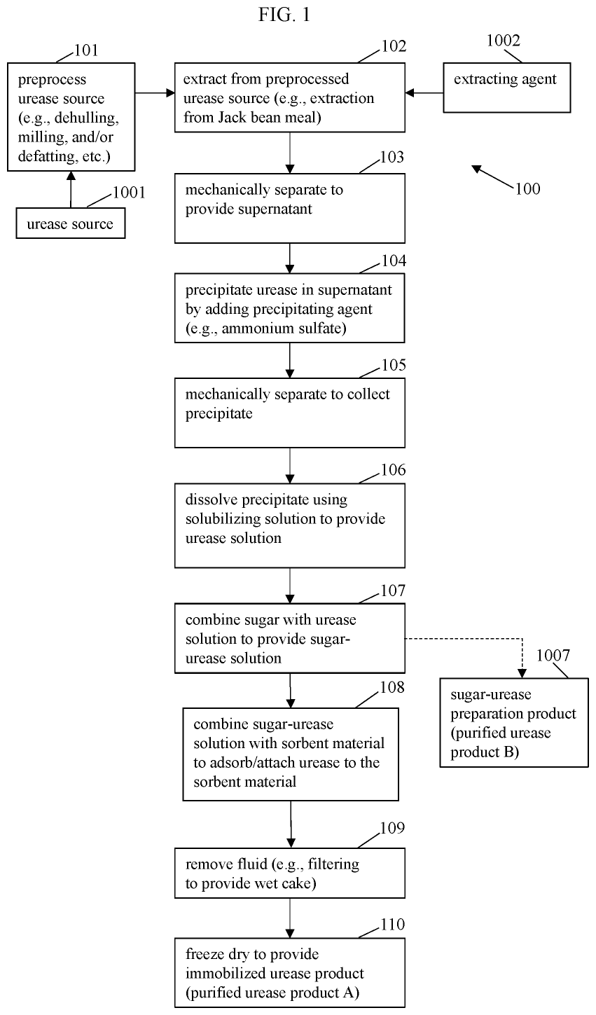

Referring to FIG. 1, a process according to an example of the present application, indicated by the identifier 100, can include at least steps 103, 104, 105, and 106, and steps 101, 102, 107, 108, and 109 are additional steps that may be included individually or in combinations thereof, depending on the starting material, choice of purified urease product, or both.

In step 101, as an option, a source of urease 1001 can be preprocessed before extracting step 102. The raw urease source, as an option, can be comminuted to reduce the size of material and increase the exposed surfaces of the material before further processing in a method of the present invention. Comminution, as used herein, refers to a process in which solid-containing materials, such as urease sources, are reduced in size. The source of urease can be, for example, ground, milled, crushed, macerated, or combinations of these and/or subjected to other attritive methods. Jack beans can be used as the urease source material but, as indicated, the method is not limited thereto. For step 101, as an option, Jack beans can be dehulled and ground (e.g., milled) into Jack bean meal. The term “Jack bean meal”, as used herein, refers to Jack beans that have been ground to fine particles. The term “hull,” as used in relation to plant seeds, refers to the dry outer covering of the seeds. As another option, the raw urease source 1001 can be directly used in the extracting step 102.

As a further option for step 101, Jack bean meal or other fat-containing urease source materials can be defatted before extracting step 102. To remove the fat from the Jack bean meal or other urease source material, the Jack bean meal can be placed in ice-chilled acetone with stirring. The Jack bean meal can be agitated in the cold acetone to allow for full or essentially full dissolution of the fat content without dissolving urease in any significant amount, and the resulting mixture can then be filtered, and the remaining (undissolved) defatted Jack bean meal can be directly used for the extracting step 102, or the defatting procedure may be repeated one or more times before using the defatted Jack bean meal in the extracting step. Other organic solvents instead of acetone may be used for defatting, such as hexane, heptane, ethanol, or others, as long as they can be used to selectively dissolve fats instead of urease.

In step 102, urease is extracted from the urease source, directly or after preprocessing. As an option, Jack bean meal can be extracted with a buffer solution which contains or is an extracting agent 1002. The extracting agent solubilizes urease such that an extract mixture is formed containing urease in solution and solids. For extraction of urease, the buffer solution can be citrate buffer solution, acetate buffer solution, phosphate buffer solution, or other buffered solutions with or without salt solution (e.g., sodium chloride) which can solubilize urease present in the urease source. The buffer should not be subject to either enzymatic or non-enzymatic changes in the presence of urease, nor should the buffer denature or otherwise chemically modify or adversely interact with urease. For extraction of urease from Jack bean meal, for example, Jack bean meal may be mixed with citrate buffer solution and stirred/agitated at about room temperature (e.g., about 20-30° C.) for thirty or more minutes, e.g., 30-120 minutes, or 30 to 90 minutes, or 30 to 40 minutes, or other durations. As an option, citrate buffer solution and Jack bean meal are blended for this extraction step, wherein from about 1000 mL to 1100 mL of citrate buffer solution per about 25 g to about 50 g Jack bean meal are combined. The term “citrate” can refer to any of their salts with citric acid, which are anhydrous or hydrated. As an option, citrate buffer solution used as the extracting agent can comprise an aqueous solution which contains citric acid and at least one of sodium citrate, potassium citrate, calcium citrate, or any combination thereof. Sodium citrate, for example, can refer to any of trisodium citrate, sodium citrate dihydrate, or sodium citrate pentahydrate.

The citrate buffer, as an option, can be at any pH of from about 5.0 to about 8.0.

As an option, the citrate buffer solution may be a 25 mM, pH 5.4 citrate buffer prepared by adding 4.95 g sodium citrate dihydrate and 1.57 g citric acid to de-ionized water and diluted to 1 L, or by mixing 25 mM sodium citrate dehydrate and 25 mM citric acid (vol:vol) of about 67:33. The citrate buffer at different pH (5.0 to 6.2) may be prepared by mixing 25 mM sodium citrate dehydrate and 25 mM citric acid (vol:vol) of about 55:45 to 80:20. Other buffer or extracting solutions for Jack bean meal or other preprocessed urease sources which may be used, including, for example, sodium acetate and sodium chloride, such as 15 mM sodium acetate solution containing 50 mM sodium chloride. For use of alumina as sorbent material in later step 108, as an option, the extracting solutions used in this step 102 can preferably be performed using sodium acetate solution containing sodium chloride.

In step 103, the resulting extract mixture from step 102 is mechanically separated to provide a supernatant which contains urease. “Mechanical separating”, as used herein, refers to any method of separating a liquid from a solid, including, e.g., centrifuging, filtrating, or decanting. As one example, the “mechanically separating” is a solid-liquid separation technique done in the absence of filtering material, e.g., solid-liquid separation is used that excludes press-filtering. A “supernatant” liquid, as used herein, refers to a liquid lying above a solid residue, such as in the extract mixture, which is separable from the solid residue by mechanical separation. As an option, the centrifuge can be a bench-top centrifuge. Any commercially-available centrifuge or centrifuging technique can be used herein. For instance, a bench-top centrifuge, as an option, can be used for solid-liquid separation on the extract mixture. As an example, the centrifuge, e.g., bench-top centrifuge can be operated at from about 3800 G to about 4000 G, for instance, at a temperature of from about 20 to about 25° C., and for instance for about 15 minutes to about 20 minutes. Other G forces, temperatures, and/or times can be used.

In step 104, urease can be precipitated in the supernatant obtained in step 103 by addition of a precipitating agent. Though not desiring to be bound by theory, at higher concentrations of precipitating agent, urease solubility usually decreases, leading to precipitation. This effect is alternatively referred to herein as salting-out. The precipitation of urease from the supernatant can be done by addition of chemical agents, referred to herein as precipitating agents. Salting out of urease from solutions can be done by addition of compounds such as ammonium sulfate ((NH₄)₂SO₄), sodium sulfate, sodium chloride, sodium monobasic phosphate, sodium dibasic phosphate, sodium acetate, or others, or any combinations thereof. In general, salts that form ions (both cation and anion) high in the Ho ineister series, and which also have high water solubility, are preferred as the precipitating agent. The precipitating agent should not be subject to either enzymatic or non-enzymatic changes in the presence of urease, nor should the precipitating agent denature or otherwise chemically modify or adversely interact with urease.

As an option, the precipitating agent is ammonium sulfate (e.g., added in solid form such as a dry powder) to a solution which contains solubilized urease, such as the supernatant obtained from step 103. The percentage of the salt used is in comparison to the maximal concentration of the salt which can dissolve in the mixture (i.e., saturation). As such, although high concentrations are desirable, adding an abundance of the salt, over 100%, can also oversaturate the solution and contaminate the precipitate. For the option of ammonium sulfate as the precipitating agent, the ammonium sulfate can be added to the supernatant obtained from step 103 in an amount of from about 40% to about 60% saturation, or from about 45% to about 55% saturation, or from about 47% to about 53% saturation, or other amounts. As an option, after an extractant of milled Jack beans in citrate buffer is centrifuged or otherwise mechanically separated in the previous step 103, solid ammonium sulfate can be added into the supernatant of the extract mixture in an amount of from about 47% to about 53% saturation to optimally precipitate urease while minimizing the precipitation of other proteins. This salting-out can assist in purifying the urease by removal of unwanted proteins and other biomolecules, such as the allergen Concanavalin A. Since this precipitation of urease is a result of a reduction in solubility rather than protein denaturation, the resulting precipitated protein material can be mechanically separated and solubilized through the use of buffers.

In step 105, the total mixture (i.e., precipitate and remaining solution) resulting from the previous salting-out step 104, is mechanically separated for solid-liquid separation. The mechanical separation for this step can be by centrifugation, decanting, or filtering or other solid-liquid separation techniques. As an option, the mechanical separation used excludes press-filtering. As an option, the total mixture is centrifuged and a solid pellet from the operation is collected. A bench-top centrifuge or other centrifuge, as an option, can be used for solid-liquid separation on the total mixture. The centrifuge can be operated at from about 3800 G to about 4000 G at a temperature of from about 20 to about 25° C. for about 50 minutes to about 60 minutes. At this scale of operation, as an option, the collected precipitate from this step can contain at least 3 grams of urease-containing protein pellet, or at least 3.2 grams of urease-containing protein pellet, or at least of 3.5 grams of urease-containing protein pellet. As an option, based on the collected pellet from step 105 compared to the supernatant obtained from step 103, from about 65% to about 75% (by weight) or higher values of non-urease contaminant proteins can be removed by the ammonium sulfate precipitation step 104. The pellet may contain at least about 25 wt % urease, or at least about 50 wt % urease, or at least about 60 wt % urease, or at least about 70 wt % urease, or at least about 80 wt % urease, or at least about 90 wt % urease, or other urease concentrations, based on total weight of proteins therein.

In step 106, the pellet collected from step 105 is dissolved or redissolved in solution containing a solubilizing agent. As an option, the solubilizing agent comprises citrate solution or other buffer solution described for step 102. As an option, the citrate solution used for this pellet dissolution step may be the same as the citrate buffer solution used for the extracting step or different therefrom. As an option, the citrate buffer solution may be a 25 mM, pH 5.4 citrate buffer prepared by adding 4.95 g sodium citrate dihydrate and 1.57 g citric acid to de-ionized water and diluted to 1 L, or by mixing 25 mM sodium citrate dehydrate and 25 mM citric acid in a vol:vol ratio of about 67:33 (i.e., Vol of sodium citrate/Vol of citric acid ratio=2.03:1). The citrate buffer at different pH (5.0 to 6.2) may be prepared by mixing 25 mM sodium citrate dehydrate and 25 mM citric acid (vol:vol) of about 55:45 to 80:20. Other buffer or dissolution solutions may be used, which can include, for example, sodium acetate solution containing sodium chloride, such as 7.5 mM sodium acetate solution containing 25 mM sodium chloride. For use of alumina as the sorbent material in later step 108, as an option, the dissolution solutions used in this step 106 can preferably be performed using sodium acetate solution containing sodium chloride.

In step 107, the urease-containing solution obtained from the pellet dissolution in previous step 106 has sugar added. The sugar, as an option, can be a monosaccharide, disaccharide, and/or oligosaccharide (i.e., a saccharide containing less than 6 monosaccharides), or any combination thereof. As an option, the sugar is at least one of glucose, galactose, fructose, ribose, pentose, hexose, sucrose, lactose, maltose, or any combination thereof. The sugar can be added to the urease-containing solution in dry dissolvable form, or as a separate sugar-containing solution. As an option, the sugar is added to the urease-containing solution to provide a sugar content of 2.5% to about 20% (w/v), or 3% to about 18% (w/v), or 4% to about 17% (w/v) or from about 5% to about 15% (w/v), Where the immobilized urease product is used in a sorbent cartridge or similar device, the sugar can be released from the immobilized urease product, such as by rinsing during priming of the sorbent cartridge with an aqueous fluid.

The immobilized urease product, as an option, can contain less than about 5%, or less than about 3%, or less than 1% by weight total moisture content.

As indicated, it has been found that the use of the sugar-urease solution can unexpectedly increase efficiency of urease recovery from 15-20% and up to as high as 65-70% during the freeze drying step. As an option, introduction of sugar into the urease-containing solution upstream of lyophilization in the indicated process increases efficiency of urease recovery during the freeze drying step, such as by about 25% or more, or any of the other indicated amounts above, compared to the process without providing sugar addition to the urease-containing solution.

The enzymatic activity (U/unit mass) of the urease which is incorporated into the immobilized urease product can be at least about 5% higher, or at least about 10% higher, or at least about 20% higher, or at least about 30% higher, or at least about 40% higher, or at least about 50% higher, or at least about 60% higher or at least about 70% higher, or from about 5% to about 70%, or from about 5% to about 50%, or from about 10% to about 60%, or from about 10% to about 45%, or from about 15% to about 40%, or from about 20% to about 35% higher, compared to the urease obtained by a method without the addition of the sugar to the urease-containing solution.

As an option, the method shown in and described herein for FIG. 1 includes at least steps 102, 103, 104, 105, 106, 107, 108, 109 and 110. As another option, the method shown in and described herein for FIG. 1 can include steps 102, 103, 104, 105, 106 and 107 (i.e., up to sugar addition to the urease solution), and one or more of steps 108, 109, and 110 (i.e., inclusive inter alia of urease immobilization on sorbent) are optional. As another option, the method shown in and described herein for FIG. 1 omits the sugar addition step 107 and retains the steps 102, 103, 104, 105, 106, 109, and 110 (i.e., inclusive inter alia of centrifugation (non-filter press/membrane based solid-liquid separation), ammonium sulfate precipitation, and urease immobilization on sorbent). As another option, the method shown in and described herein for FIG. 1 includes at least steps 104, 105, 107, and 108.

Purified urease products according to examples of the present application can be arranged or packed inside sorbent holding structures, such as sorbent cartridges, which will retain the purified urease product in position while flow of fluid is conducted into and out of the device with passage through the urease product, e.g., one or more layers of the purified urease product. The sorbent cartridge(s) described here is/are comprised of at least one layer comprising a purified urease product according to an example of the present application. Urease is included in the sorbent cartridges of the present application to provide a catalytic effect with respect to urea in aqueous fluids treated by the sorbent cartridge. For use in hemodialysis systems, the sorbent cartridges are preferably comprised of different layers of highly specified and designed materials, and perform the regenerative function by employing three chemical phenomena: (i) adsorption, (ii) catalysis, and (iii) ion exchange. Adsorption describes the immobilization or fixation of mobile species at a solid interface or surface. Catalysis, such as the catalytic effect of urease on urea in aqueous fluid being treated, is a process by which the rate of a chemical reaction is increased by the reduction of the reaction activation energy via a component in the reaction whose net rate of consumption is zero. Ion exchange is a process in which particular solid materials adsorb species for which they have a high affinity and in turn release a species for which its affinity is lower.

In accordance with the techniques described herein, and with no limitation on the layer chemistry other than including at least one layer which comprises the purified urease product, a sorbent cartridge can be provided that can include a housing and optionally one or more other sorbent layers in addition the layer comprising the purified urease product. The housing can define a cartridge interior, the cartridge interior having a volume and configured to hold at least one layer of sorbent material. The housing can include a first end having a first port configured to permit entry of a fluid into the cartridge interior, and a second end distal to the first end and having a second port configured to permit exit of the fluid from the cartridge interior. One will appreciate that the techniques described herein need not be dependent on a particular housing or housing configuration, and that the housing is provided as a conventional way to hold and contain various sorbent layers, as well as effluent passing through the layers.

As indicated, at least one layer of the sorbent cartridge is sorbent material which comprises the purified urease product, for example, in the form of the freeze dried immobilized urease product. The purified urease product can be used in a single layer or multiple layers in the sorbent cartridge. Purified urease used in different layers in the same cartridge can have the same or different specific activity between the two layers.

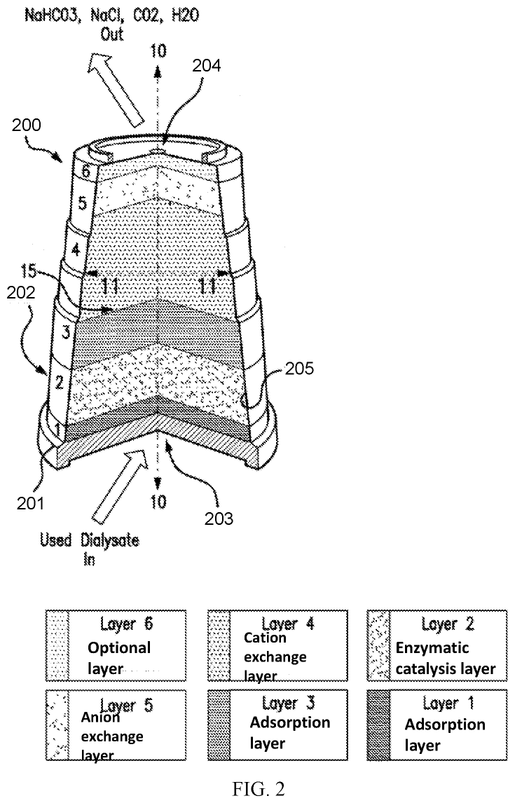

FIG. 2 shows a sorbent cartridge of an example of the present application that is being used for treatment of dialysate fluid. The sorbent cartridge is identified in FIG. 2 as component 200, which has a housing 201, which comprises a solid continuous sidewall 202, a continuous inner sidewall 205, inlet end wall 203, and outlet end wall 204, and a multi-layered sorbent bed 15 is incorporated within the housing 201. The sorbent bed 15 is shown here comprised of layers 1-6 and a central longitudinal axis 10-10, which extends through the sorbent bed 15 (usually coinciding with or near the geometric center of sorbent bed layers 1-6). As an option, layer 1 is an adsorption layer, layer 2 is an enzymatic catalysis layer, layer 3 is another adsorption layer, layer 4 is a cation exchange layer, and layer 5 is an anion exchange layer. Layer 6 is an optional layer. As an option, in sorbent cartridge 200, layer 1 comprises activated carbon, layer 2 comprises the purified urease (e.g., a layer of the immobilized urease product according to an example of the present application), layer 3 comprises activated carbon, layer 4 comprises zirconium phosphate, and layer 5 comprises zirconium oxide (e.g., a hydrous zirconium oxide layer). Optional layer 6, if included, can comprise sodium bicarbonate, sodium zirconium carbonate, granular activated carbon, fillers, or other materials. These layers each form a distinct stratum of the overall particle bed. Additional, different, or less layers can be included in the sorbent bed in the cartridge.

The sorbent bed layers 1-5 (and 6, if included) can extend in directions 11 radially outward (and usually orthogonally or substantially orthogonally (e.g., within 1 to 10 degrees of orthogonal)) from the central longitudinal axis 10-10 to an inner face (wall) 205 of the continuous sidewall 202 of the housing 201. In this configuration, as an option, each of layers 1-5 (and 6, if included) of the sorbent cartridge can be comprised of material of similar chemical composition and physical properties per layer (e.g., particle size distribution, morphology, crystallinity and/or other properties). The particles used in these layers can be originally supplied in freely flowable solid particulate form. Once incorporated into the respective layers in the cartridge, they are packed into layered beds comprising strata formed of the particles. In the sorbent bed 15 shown in FIG. 2, the layers 1 and 3-5 (and 6, if included) containing non-urease materials are shown here for sake of illustration and the concepts described herein are not at all limited to these layers or types of layers for use in combination with the layer comprising the purified urease.

Non-limiting examples of the sorbent cartridges are further discussed as follows. Each of these examples can include a housing that surrounds all or a portion of the sorbent layers. The housing can conform to the shape of the sorbent layers in whole or part, or can be independent of the sorbent layer profile. Sorbent layers can be formed using any desired technique. For example, solid molds or hollow frames can be used to form the various strata (horizontal slices) and sorbent layers of a given sorbent cartridge. Sorbent layers of a given stratum can be formed simultaneously or in stages, for example, for successive concentric or nested sorbent layers. Adjacent sorbent layers can have sharp, distinct, blurred, and/or transitioned boundaries. Sorbent layers can contain gradients of sorbent material with respect to density, surface area, composition, and/or any other desired characteristic or combination of characteristics. The shape, size, order, and/or number of the strata and/or layers can vary as desired. Sorbent layers and/or strata can include any shapes or combination of shapes, curvilinear and/or rectilinear, for example, cones, cylinders, conical frustums, polygonal (regular and/or irregular) frustums, cylindrical prisms, conical prisms, polygonal (regular and/or irregular) prisms, and the like. The sides of a sorbent cartridge can be continuous or discontinuous, smooth or stepped, or a combination thereof; a description of one is understood to be representative of the other. Descriptions of square embodiments are also representative of rhombic, rectangular, regular polygonal, and irregular polygonal embodiments, and the like. For instance, though the sorbent cartridge in FIG. 2 is shown with a tapered shaped sidewall, which has a diameter that smoothly tapers inward towards the outlet end, the indicated concepts described herein can be applied to cartridges that have other shapes, such as cylindrical, rectangular (e.g., square), hexagonal, or other shapes. Any geometric shape can generally be used. While strata generally refer to horizontal slices, other orientations are also encompassed by the techniques described herein.

As an option, it is possible to prepare a layer or multiple layer arrangement of sorbent(s) and insert this arrangement into a housing afterwards. The layer arrangement can be provided in a way that it can be inserted into a cartridge or housing or other holding structure at any time or right before using. The layer arrangement can be structurally kept in place by temporary molds (e.g., paper, plastic, and the like). The sorbent bed can include a multilayer stack which comprises at least the first and second layers, wherein the multilayer stack is insertable into a sorbent cartridge housing. All of the options, details, discussion above regarding the layers and the like equally apply here to this aspect of the present invention.

As indicated, the techniques described herein, as an option, can relate to a sorbent cartridge, such as shown in FIG. 2 that includes at least dialysate treatment components of carbon, purified urease product, zirconium phosphate (“ZP”), and zirconium oxide. Other components may be used in combination with the purified urease product in various arrangements in a sorbent cartridge.

The order and composition of layers for a cartridge design of the present invention prior to be used to regenerate or purify spent dialysis fluid, can be, for example, as follows (e.g., top (exit or outlet) to bottom (entrance-inlet) in the cartridge):

a) one or more layers comprising, consisting essentially of, consisting of, or including hydrous zirconium oxide-hydroxide and/or hydrous zirconium oxide-chloride (e.g., 150 g to about 250 g),

b) one or more layers comprising, consisting essentially of, consisting of, or including zirconium phosphate (e.g., 650 g to about 1800 g), for instance, with a sodium loading of from about 50 mg to about 56 mg Na/g zirconium phosphate (the zirconium phosphate can have the formula as set forth in the Background above),

c) one or more layers comprising, consisting essentially of, consisting of, or including a carbon layer or pad (e.g., about 50 g to about 500 g carbon),

d) one or more enzyme containing layers, such as a layer comprising, consisting essentially of, consisting of, or including purified urease product, for example, as immobilized urease product (e.g., lyophilized silica gel-urease) (e.g., about 150 g to about 300 g immobilized urease product, including from about 37000 I.U. to about 55000 I.U. of urease activity), and

e) one or more layers comprising, consisting essentially of, consisting of, or including a carbon layer or pad (e.g., about 50 g to about 500 g carbon). These amounts for components a)-g) are provided as an example, and other amounts of these materials may be used. As used herein, a urease enzyme unit is defined as follows: one unit will liberate 1.0 μmole of ammonia from urea per minute at pH 7.0 at 25° C. It is equivalent to 1.0 I.U.

Referring to FIG. 2, as an option, for a sorbent cartridge which can be used in the present invention, hydrous zirconium oxide-chloride which has an alkaline pH can be used in layer 5 in an amount of from about 50 g to about 300 g, or from about 75 g to about 200 g, or about 100 g, or other amounts. The zirconium phosphate in layer 4 can be used in an amount of from about 650 g to about 1800 g, or from about 800 g to about 1600 g, or from about 900 g to about 1300 g, or other amounts. The zirconium phosphate of this example can have a sodium loading of greater than 55 mg/g Na/g zirconium phosphate, or from about 56 mg to about 58 mg Na/g ZP, or about 57 mg Na/g ZP, or other values. The carbon layer or pad 4 can be used in an amount of from about 50 g to about 500 g carbon or other amounts. The purified urease product, such as present as immobilized urease product, in layer 2 can be used in amounts of from about 150 g to about 300 g immobilized urease product, including from about 37000 I.U. to about 74000 I.U., or from about 37000 I.U. to about 55000 I.U., of urease activity or other units of urease activity. The bottom carbon layer or pad 1 can be used in an amount of from about 50 g to about 500 g carbon or other amounts. Any effective amounts of the above-described materials can be present in the cartridge. These amounts (or any amounts recited herein) can be with respect to a cartridge having the following dimensions: 2 inches-3 inches diameter by 5 inches to 10 inches length, or having the following dimensions: 4 inches-6 inches diameter by 6 inches-12 inches length. However, it is to be understood that these amounts provide weight ratios for each layer with respect to each other layer so as to permit adjustments in any sized cartridge.

Additional options for the indicated materials other than the purified urease product which may be used in other layers of the sorbent cartridge, such as for the carbon, zirconium phosphate, zirconium oxide, and sodium bicarbonate, can include those disclosed in U.S. Patent Application Publication Nos. US 2017/0189598 A1, US 2010/0078387 A1 and US 2006/0140840 A1, and U.S. Pat. No. 6,627,164 B2, which are incorporated in their entireties by reference herein. Other sorbent cartridge materials, amounts, and other optional components and/or dialysis systems which may be used are described in the following patents and publications can also be used in the present application and are incorporated in their entirety by reference herein and form a part of the present application: Des. 282,578; 3,669,878; 3,669,880; 3,697,410; 3,697,418; 3,703,959; 3,850,835; 3,989,622; 3,989,625; 4,025,608; 4,213,859; 4,256,718; 4,360,507; 4,460,555; 4,484,599; 4,495,129; 4,558,996; 7,033,498 B2, and the following articles, “Guide to Custom Dialysis,” Product No. 306100-005, Revision E, pages 1-54, dated September 1993 and “Sorbent Dialysis Primer,” Product No. 306100-006, Edition 4, pp. 1-51, dated September 1993 of Cobe Renal Care, Inc.

Further, a single cartridge can be used which combines all of the above-described materials. In another example, a series of cartridges can be used wherein the combination of the above-described materials can be present in one or more cartridges. For instance, purified urease product and split carbon layers that sandwich this layer can be provided in a first cartridge and the remaining layers can be placed in a second cartridge, and so on. Optionally, these various indicated layers in these sequences can be divided over three different cartridges or more. As indicated, all of the materials can be provided in a single cartridge and can be arranged as distinct layers in the single cartridge. As an option, a cartridge layer can be composed of at least about 50% by weight, or at least 75% by weight, or at least about 80% by weight, or at least about 90% by weight, or at least about 95% by weight, or least about 99% by weight, or up to 100% by weight, or from about 50% to about 100% by weight, or from about 75% to about 100% by weight, or from about 90% to about 100% by weight, or from about 95% to about 100% by weight, or from about 99% to about 100% by weight, of only the material or materials indicated for use in that layer.

As an option, in addition to any carbon filter pad that may be used in providing one or both of the indicated carbon layers on each side of the enzyme containing layer, one or more filter pads can be located throughout the sorbent cartridge to ensure that the layer integrity is maintained during operation. The filter pad can be made of any type of material, for instance, standard filter paper or cellulose pads and the like and typically is the diameter or length-width of the cartridge in order to separate completely one layer from another layer. A flow diffuser which uniformly diffuses the used dialysate throughout the entire width or diameter of the sorbent cartridge can be used. The flow diffuser can have a design of radial spreading channels made of plastic or other suitable materials. The flow diffuser is typically located prior to any of the optional filter pads or materials used in the sorbent cartridge and is adjacent to the inlet (or part of the inlet) of the sorbent cartridge. A barrier layer(s) can also be used in the sorbent cartridge. A barrier layer can be located between the immobilized enzyme layer and the alumina layer, if present. An example of a barrier layer includes filter paper and the like.

Various overall shapes of the sorbent cartridge include, but are not limited to, a cylindrical shape, rectangular shape, a pyramidal-cylindrical (stepped) shape as shown, for instance, in FIG. 2 and so on. The shape can be straight-edged or tapered, and so on. Any geometric shape can generally be used. As an option, a peritoneal dialysis (PD) cartridge can have the following dimensions: 2 inches-3 inches diameter by 5 inches to 10 inches length. A hemodialysis (HD) cartridge can have the following dimensions: 4 inches-6 inches diameter by 6 inches-12 inches long. Other dimensions can be used depending on the needs of the purifying, amount to purify, operating system and the like. Examples of cartridge designs are further shown in U.S. Pat. No. 6,878,283, which is incorporated in its entirety by reference herein. Examples of cartridges are also described in one or more of the patents and/or publications identified herein.

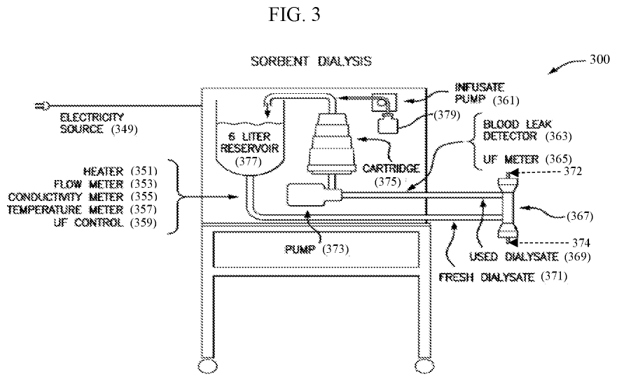

As described in more detail below, the sorbent cartridges as described herein can be used in a variety of separation systems and can be used in the regeneration or purification of dialysates (e.g., HD) or PD solutions. For purposes of the present invention, a dialysis solution means a peritoneal dialysis solution or dialysate fluids that are useful in hemodialysis or sorbent dialysis systems. Conventional dialysis solutions for PD or HD can be used and regenerated by the present invention and are known to those skilled in the art. In a less complicated design, spent or used dialysate or PD solutions can simply be passed through one or more cartridges to purify or regenerate the spent fluids. Such a system can be straightforward in setup and can involve merely using a column-type setup wherein the spent fluids are passed from top to bottom wherein gravity permits the spent fluid to go through the cartridge or spent fluid can be passed through the cartridge under pressure which permits the spent fluids to be introduced in any direction. In a more specific system, the system set forth in FIG. 3, and identified by numeral 300, can be adapted to use an indicated sorbent cartridge as used especially for hemodialysis; that is a system that can be used as a closed system, or alternatively in a single pass dialysis system (not shown). Such a system permits the continuous reusing of the regenerated dialysate in a patient during dialysis treatment. With respect to a single pass system (not shown), in lieu of discarding the used dialysate to a floor drain, as an alternative, the used dialysis can simply be collected in a container which then can be regenerated or purified by passing the spent dialysate through one or more cartridges as described above.

With respect to peritoneal dialysis, there are several options. First, like hemodialysis, the peritoneal dialysis solution that is spent can be directly passed through one or more cartridges to purify or regenerate the used peritoneal dialysis solution in order to remove the waste products. Alternatively, the peritoneal dialysis solution which is used or spent can first be passed through a dialyzer in the same manner as blood during hemodialysis wherein dialysate removes waste products and the like from the peritoneal dialysis solution and then the dialysate can be regenerated or purified by passing the used or spent dialysate through the cartridge. Either system can be used. With a closed PD system, the risk of peritonitis can be reduced since the frequent connections made with conventional systems between the catheter in the peritoneal cavity and a succession of dialysis solution containers can be avoided.

Referring to FIG. 3, a sorbent dialysis system identified as 300 is shown which includes a sorbent cartridge 375, which is a sorbent cartridge of the present application. 349 refers to a source of electricity to operate the dialysis system. 351 represents a heater, 353 represents a flow meter, 355 represents a conductivity meter, 357 represents a temperature meter, and 359 represents a UF control. These items (349, 351, 353, 355, 357, 359, and a sorbent cartridge more generally) are conventional items in a sorbent dialysis system and are known to those skilled in the art and can be used in implementing the techniques described herein. 361 is an infusate pump that is used to pump in fresh concentrate 379 to be mixed with the regenerated dialysate which ultimately enters the reservoir 377, which can be, e.g., a six liter reservoir. 363 represents a blood leak detector and 365 represents a UF meter which are conventional items in dialysis systems and can be used herein. Component 367 represents a dialyzer. To simplify this illustration, the arterial and venous blood lines 372 and 374, which ultimately extend to and from a patient undergoing dialysis treatment and the supporting blood pump(s) and other associated elements usually used therewith, are not fully shown. As indicated, a dialyzer is known by those skilled in the art and typically is a system or component that contains a membrane in order to have the waste products pass from the blood through the membrane to the dialysate fluid. Similarly, 369 represents used dialysate leaving the dialyzer and 371 represents fresh or regenerated dialysate entering the dialyzer 367. Component 373 is a pump to pump the used dialysate from the dialyzer into the sorbent cartridge 375 which are the cartridges of the present application.

The sorbent cartridges described herein can be made for use in multiple hours of dialysis treatment, such as, for example, for up to about 4 hours of dialysis treatment or for up to about 8 hours of dialysis treatment. For example, the 8 hour cartridges can typically be made for home use and the 4 hour cartridges can typically be made for dialysis treatment in medical treatment or dialysis centers. The cartridges described herein can generally be used with any type of dialysis system as described above. The flows that pass through the cartridge are typically any conventional flows. For instance, flows from about 50 ml/min or less to 500 ml/min or more of dialysate can flow through the cartridge and can be used in the systems described herein. Other flows can be used depending upon the size of the cartridge and the operating system.

The dialysis systems or components thereof described in the above and following patents can be used in the present application and these systems can incorporate the materials and/or cartridges described herein: U.S. Pat. Nos. 7,033,498 B2; 8,663,463; 8,597,505; 8,580,112; 8,500,994; 8,366,921; 8,343,346; 8,475,399; 8,012,118; and 9,867,918. All of these patents and patent applications are incorporated in their entirety by reference herein and form a part of the present application.

There are numerous uses for the materials described herein and especially the cartridges described hereinsuch as the regeneration of dialysis fluids as mentioned above. Furthermore, the cartridges can also be used in any separation process which requires the removal of urea alone or along with other impurities or waste products from a fluid or other medium that is passable through the materials of the present invention. Also, the techniques described herein may be useful with respect to treating drug overdose patients or other patients which are in need or removing undesirable or dangerous contaminants in a person's blood stream.

Accordingly, the techniques described herein provide useful embodiments that allow the regeneration of dialysate type fluids and other fluids.

The techniques described herein can be used to provide stationary sorbent dialysis systems or portable sorbent dialysis systems. The sorbent dialysis systems can include sorbent hemodialysis, a wearable artificial kidney, sorbent peritoneal dialysis, and other sorbent dialysis systems.

The purified urease products and sorbent bed and cartridges of the present invention also can be used in wastewater treatment and other uses. Urea is one of the major products of mammalian protein metabolism, which leads to its presence in municipal sewer water and in natural bodies of water from runoff. Urea is also industrially produced in large quantities. Urea thus can enter into the environment not only with wastewater from the production plants but also by leaching from the fields, farms, and in effluents from plants using urea as a raw material. The urease products and sorbent bed and cartridges of the present invention can be used in treatment of wastewaters, such as from one or more of these sources which contain urea.

The present invention will be further clarified by the following examples, which are intended to be only exemplary of the present invention. Unless indicated otherwise, all amounts, percentages, ratios and the like used herein are by weight.

### EXAMPLES

### Example 1

Laboratory experiments were conducted to study urease purification according to a method of the present invention and a comparison example.

Jack beans were milled to provide Jack bean meal. 25 g Jack bean meal was mixed with 1000 mL citrate buffer (25 mM, pH 5.4) with stirring in a laboratory beaker at room temperature for about 30 minutes. The citrate buffer was prepared by adding 4.95 g sodium citrate dihydrate and 1.57 g citric acid to de-ionized water and diluted to 1 L. The extract mixture was centrifuged in a bench-top centrifuge at 3804 g at 25° C. for about 15 minutes. 31.38 g solid ammonium sulfate per 100 mL of supernatant was added incrementally with stirring at room temperature for about 30 minutes. The protein solution was centrifuged in a bench-top centrifuge at 3804 G at 25° C. for about 50 minutes to separate a solid pellet (approx. 3.5 g) from the liquid. The collected pellet redissolved in 350 mL of the citrate buffer. Sucrose in dry powder form was added to the solution to reach about 5% (w/v) sugar content therein. The resulting sugar-urease solution (375 mL) was mixed with 94 g silica gel sorbent material with M:V (Mass to Volume) ratio of 1:4 to adsorb and immobilize the urease enzyme on the silica gel. The silica gel particle size was from about 40 μm to 75 μm, and had an average pore diameter of 800 Å to about 1000 Å. The resulting slurry containing urease-silica gel composite material was filtered using a Buchner funnel with a 1.6 g glass fiber filter paper. The recovered sorbent-urease wet cake was lyophilized in a freeze dryer at a temperature of −30° C. and a vacuum of 50 mtorr for about 10-15 hours followed by a secondary drying at 0° C. and 100 mTorr for about 3 hours to produce an immobilized urease product with moisture content (by wt %) less than 5%, which can be used in sorbent cartridges or other purification devices used for urea removal from aqueous fluids. Similar runs were conducted at other sucrose addition levels in the range of 5-15% (w/v).

### Example 2

Additional laboratory experiments were conducted to study urease purification according to a method of the present invention and a comparison example, wherein alumina was used as sorbent material.

Jack beans were milled to provide Jack bean meal. 25 g Jack bean meal was mixed with 1000 mL salt solution containing 15 mM sodium acetate and 50 mM sodium chloride with stirring in a laboratory beaker at room temperature for about 30 minutes. The salt solution was prepared by adding 2.15 g sodium acetate trihydrate and 2.9 g sodium chloride to de-ionized water and diluted to 1 L. The extract mixture was centrifuged in a bench-top centrifuge at 3804 G at 25° C. for about 15 minutes. 31.38 g solid ammonium sulfate per 100 mL of supernatant was added incrementally with stirring at room temperature for about 30 minutes. The protein solution was centrifuged in a bench-top centrifuge at 3804 G at 25° C. for about 50 minutes to collect the soft urease-containing protein pellet (approx. 4.8 g). The collected pellet was redissolved in 480 mL of the diluted salt solution containing 7.5 mM sodium acetate and 25 mM sodium chloride. Sucrose was added to the solution with stirring to reach about 5% (w/v) sugar content therein. The sucrose was added in dry powder form. The resulting sugar-urease solution (510 mL) was mixed with 170 g of alumina sorbent material with M:V ratio at 1:3, which was a commercial product with particle size about 40 μm to about 150 μm to adsorb and immobilize the urease enzyme on the silica gel. The resulting slurry containing urease-silica gel composite material was filtered using a Buchner funnel with a 1.6μ glass fiber filter paper. The recovered sorbent-urease wet cake was lyophilized in a freeze dryer at a temperature of −30° C. and a vacuum of 50 mtorr for about 10-15 hours followed by a secondary drying cycle at 0° C. for about 3 hours to produce an immobilized urease product with moisture content (by wt %) less than 5%, which can be used in sorbent cartridges or other purification devices used for urea removal from aqueous fluids. Similar runs were conducted at other sucrose addition levels in the range of 5-15% (w/v).

For comparison, the indicated method was repeated to produce an immobilized urease product without including the sugar addition step.

For these test runs, urease recovery efficiency (%) based on enzymatic activity was determined for each run by enzymatic assay of urease.

The results from Examples 1 and 2 show that the use of 5-15% (w/v) sucrose solution unexpectedly increases efficiency of urease recovery from 15-20% (without sucrose addition) to 65-70% (with sucrose addition) during the freeze drying step.

The present invention includes the following aspects/embodiments/features in any order and/or in any combination:

1. The present invention relates to a method of purifying urease, comprising:

a) mechanically separating an extract mixture to provide a separated solution, wherein the extract mixture comprises an extracting agent in solution and a comminuted source of urease;

b) combining ammonium sulfate with the separated solution to precipitate urease in the separated solution to provide a precipitate-containing mixture;

c) mechanically separating the precipitate-containing mixture to collect the precipitate;

d) dissolving the precipitate collected to provide a urease-containing solution;

e) combining the urease-containing solution with a sugar to provide a sugar-urease solution;

f) combining the sugar-urease solution and a sorbent material to immobilize urease on the sorbent material to provide an immobilized urease preparation; and

g) optionally freeze drying the immobilized urease preparation to provide an immobilized urease product.

2. The method of any preceding or following embodiment/feature/aspect, wherein the source of urease is a botanical source of urease, fungal source of urease, algal source of urease, bacterial source of urease, invertebrate source of urease, or any combination thereof.
3. The method of any preceding or following embodiment/feature/aspect, wherein the source of urease comprises jack beans, sword beans, soy beans, or any combination thereof.
4. The method of any preceding or following embodiment/feature/aspect, wherein the source of urease is jack beans.
5. The method of any preceding or following embodiment/feature/aspect, further comprising milling dehulled jack beans before step a) to provide the comminuted source of urease.
6. The method of any preceding or following embodiment/feature/aspect, wherein the sugar comprises a monosaccharide, a disaccharide, or an oligosaccharide, or any combination thereof.
7. The method of any preceding or following embodiment/feature/aspect, wherein the sugar is added to the urease solution in an amount to provide from about 2.5% to about 20%, preferably from about 5% to about 15%, (w/v) sugar (e.g., sucrose) in the sugar-urease solution.
8. The method of any preceding or following embodiment/feature/aspect, wherein the extracting agent comprises citrate.
9. The method of any preceding or following embodiment/feature/aspect, wherein the ammonium sulfate is added in solid form to the separated solution in b) in an amount providing from about 40% to about 60% saturation therewith.
10. The method of any preceding or following embodiment/feature/aspect, wherein the precipitate collected from c) is at least about 25 wt % urease, based on total weight of proteins therein.
11. The method of any preceding or following embodiment/feature/aspect, wherein the mechanically separating in a), c), or both a) and c), comprises centrifuging.
12. The method of any preceding or following embodiment/feature/aspect, wherein the centrifuging comprising centrifuging using a bench-top centrifuge or production scale centrifuge.
13. The method of any preceding or following embodiment/feature/aspect, wherein the precipitate is dissolved in citrate-containing solution in d), wherein the citrate optionally is at least one of sodium citrate, potassium citrate, calcium citrate, or any combination thereof.
14. The method of any preceding or following embodiment/feature/aspect, wherein the sorbent material comprises siliceous support material.
15. The method of any preceding or following embodiment/feature/aspect, wherein the sorbent material comprises silica gel.
16. The method of any preceding or following embodiment/feature/aspect, wherein f) further comprises i) combining the fluid mixture and sorbent material to provide a solid-liquid suspension, wherein urease is immobilized on the sorbent material in the solid-liquid suspension, and ii) removing fluid from the solid-liquid suspension to provide wet cake, and g) further comprises freeze drying the wet cake to provide the immobilized urease product.
17. The method of any preceding or following embodiment/feature/aspect, wherein the sorbent material comprises alumina.
18. The method of any preceding or following embodiment/feature/aspect, wherein the enzymatic activity (U/unit mass) of the urease contained by the immobilized urease product is from about 5% to about 50% or higher compared to the urease contained by the immobilized urease product obtained by a method without the addition of the sugar to the urease-containing solution.
19. The present invention relates to a method of purifying urease, comprising:

a) combining ammonium sulfate with an urease-solute containing solution to precipitate urease to provide a precipitate-containing mixture;

b) mechanically separating the precipitate-containing mixture to collect the precipitate;

c) combining the precipitate, sugar, and solubilizing agent in solution, in any order, to provide a sugar-urease solution; and

d) combining the sugar-urease solution and sorbent material to immobilize urease on the sorbent material to provide an immobilized urease preparation.

20. The present invention relates to an immobilized urease product prepared by the method of any of preceding or following embodiment/feature/aspect.
21. The present invention relates to an immobilized urease product comprising sorbent material, urease immobilized on the sorbent material, and sugar (e.g., about 1 ppm to about 1000 ppm sugar based on total solids weight of product), or as prepared by the method of any of preceding or following embodiment/feature/aspect.
22. The immobilized urease product of any preceding or following embodiment/feature/aspect, wherein sorbent material comprises silica gel.
23. The present invention relates to a sorbent cartridge comprising a urease-containing layer comprising the immobilized urease product of any preceding or following embodiment/feature/aspect.
24. The sorbent cartridge of any preceding or following embodiment/feature/aspect, wherein the sorbent cartridge further comprises at least one activated carbon layer, at least one cation exchange material-containing layer, and at least one anion exchange material-containing layer.
25. The sorbent cartridge any preceding or following embodiment/feature/aspect, wherein the sorbent cartridge further comprising, from the fluid inlet to the fluid outlet:

a) a first activated carbon-containing layer that precedes the urease-containing layer;

b) the urease-containing layer, which follows the first carbon-containing layer within the sorbent cartridge;

c) a second activated carbon-containing layer that follows the urease-containing layer within the sorbent cartridge;

d) a zirconium phosphate-containing layer, which follows the second carbon-containing layer within the sorbent cartridge;

e) a hydrous zirconium oxide layer that follows the zirconium phosphate-containing layer; and

f) optionally a (bi)carbonate layer that follows the hydrous zirconium oxide layer comprising sodium (bi)carbonate.

26. The present invention relates to a method to regenerate or purify dialysis fluid comprising passing dialysis fluid through a sorbent cartridge of any preceding or following embodiment/feature/aspect.
27. The present invention relates to a dialysis system to regenerate or purify spent dialysis fluid comprising the sorbent cartridge of any preceding or following embodiment/feature/aspect.

The present invention can include any combination of these various features or embodiments above and/or below as set forth in sentences and/or paragraphs. Any combination of disclosed features herein is considered part of the present invention and no limitation is intended with respect to combinable features.

Applicants specifically incorporate the entire contents of all cited references in this disclosure. Further, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

Other embodiments of the present invention will be apparent to those skilled in the art from consideration of the present specification and practice of the present invention disclosed herein. It is intended that the present specification and examples be considered as exemplary only with a true scope and spirit of the invention being indicated by the following claims and equivalents thereof.

What is claimed is:

1. A method of purifying urease, comprising:
a) mechanically separating an extract mixture to provide a separated solution, wherein the extract mixture comprises an extracting agent in solution and a comminuted source of urease;
b) combining ammonium sulfate with the separated solution to precipitate urease in the separated solution to provide a precipitate-containing mixture;
c) mechanically separating the precipitate-containing mixture to collect the precipitate;
d) dissolving the precipitate collected to provide a urease-containing solution;
e) combining the urease-containing solution with a sugar to provide a sugar-urease solution;
f) combining the sugar-urease solution and a sorbent material to immobilize urease on the sorbent material to provide an immobilized urease preparation; and
g) optionally freeze drying the immobilized urease preparation to provide an immobilized urease product.

2. The method of claim 1, wherein the source of urease is a botanical source of urease, fungal source of urease, algal source of urease, bacterial source of urease, invertebrate source of urease, or any combination thereof.

3. The method of claim 1, wherein the source of urease comprises jack beans, sword beans, soy beans, or any combination thereof.

4. The method of claim 1, wherein the source of urease is jack beans.

5. The method of claim 1, further comprising milling dehulled jack beans before step a) to provide the comminuted source of urease.

6. The method of claim 1, wherein the sugar comprises a monosaccharide, a disaccharide, or an oligosaccharide, or any combination thereof.

7. The method of claim 1, wherein the sugar is added to the urease-containing solution in an amount providing from about 2.5% to about 20% (w/v) sugar in the sugar-urease solution.

8. The method of claim 1, wherein the extracting agent comprises citrate.

9. The method of claim 1, wherein the ammonium sulfate is added in solid form to the separated solution in b) in an amount providing from about 40% to about 60% saturation therewith.

10. The method of claim 1, wherein the precipitate collected from c) is at least about 25, wt % urease, based on total weight of proteins therein.

11. The method of claim 1, wherein the mechanically separating in a), c), or both a) and c), comprises centrifuging.

12. The method of claim 11, wherein the centrifuging comprising centrifuging using a bench-top centrifuge or a production scale centrifuge.

13. The method of claim 1, wherein the precipitate is dissolved in citrate-containing solution in d).

14. The method of claim 1, wherein the sorbent material comprises siliceous support material.

15. The method of claim 1, wherein the sorbent material comprises silica gel.

16. The method of claim 1, wherein f) further comprises i) combining the fluid mixture and sorbent material to provide a solid-liquid suspension, wherein urease is immobilized on the sorbent material in the solid-liquid suspension, and ii) removing fluid from the solid-liquid suspension to provide wet cake, and g) further comprises freeze drying the wet cake to provide the immobilized urease product.

17. The method of claim 1, wherein the sorbent material comprises alumina.

18. The method of claim 1, wherein the enzymatic activity (U/unit mass) of the urease contained by the immobilized urease product is from about 5% to about 50% or higher compared to the urease contained by the immobilized urease product obtained by a method without the addition of the sugar to the urease-containing solution.

19. A method of purifying urease, comprising:
a) combining ammonium sulfate with urease-solute containing solution to precipitate urease to provide a precipitate-containing mixture;
b) mechanically separating the precipitate-containing mixture to collect the precipitate;
c) combining the precipitate, sugar, and solubilizing agent in solution, in any order, to provide a sugar-urease solution; and
d) combining the sugar-urease solution and sorbent material to immobilize urease on the sorbent material to provide an immobilized urease preparation.

20. An immobilized urease product prepared by the method of claim 19.

21. An immobilized urease product comprising sorbent material, urease immobilized on the sorbent material, and sugar.

22. The immobilized urease product of claim 21, wherein sorbent material comprises silica gel.

23. A sorbent cartridge comprising a urease-containing layer comprising the immobilized urease product of claim 21.

24. The sorbent cartridge of claim 23, wherein the sorbent cartridge further comprises at least one activated carbon layer, at least one cation exchange material-containing layer, and at least one anion exchange material-containing layer.

25. The sorbent cartridge of claim 23, wherein the sorbent cartridge further comprising, from the fluid inlet to the fluid outlet:
a) a first activated carbon-containing layer that precedes the urease-containing layer;
b) the urease-containing layer, which follows the first carbon-containing layer within the sorbent cartridge;
c) a second activated carbon-containing layer that follows the urease-containing layer within the sorbent cartridge;
d) a zirconium phosphate-containing layer, which follows the second carbon-containing layer within the sorbent cartridge;
e) a hydrous zirconium oxide layer that follows the zirconium phosphate-containing layer; and
f) optionally a (bi)carbonate layer that follows the hydrous zirconium oxide layer comprising sodium (bi)carbonate.

26. A method to regenerate or purify dialysis fluid comprising passing dialysis fluid through a sorbent cartridge of claim 23.

27. A dialysis system to regenerate or purify spent dialysis fluid comprising the sorbent cartridge of claim 23.
